(19) United States
(12) Patent Application Publication
(10) Pub. No.: US 2025/0332437 A1
(43) Pub. Date: Oct. 30, 2025

(54) METHOD AND SYSTEM FOR IRRADIATING A REGION OF THE SKIN OF A SUBJECT

(71) Applicant: PHLECS B.V., EINDHOVEN (NL)

(72) Inventors: Maarten Joost DEN HOLLANDER, EINDHOVEN (NL); Theodurus Wilhelmus Rene WENMEKERS, EINDHOVEN (NL); David Jack Albert AUBERT, EINDHOVEN (NL)

(21) Appl. No.: 18/865,662

(22) PCT Filed: Jun. 6, 2023
(86) PCT No.: PCT/NL2023/050310

§ 371 Date: Nov. 13, 2024

(30) Foreign Application Priority Data
Jun. 7, 2022 (NL) 2032084

(51) Int. Cl. A61N 5/06; G16H 40/63

(52) U.S. Cl. CPC A61N 5/0616; A61N 5/0618; A61N 5/0621; A61N 5/0622; G16H 40/63; A61N 2005/0627; A61N 2005/0632; A61N 2005/0637; A61N 2005/0642; A61N 2005/0659; A61N 2005/0661; A61N 2005/0663

(57) ABSTRACT

The present invention relates to a method of determining the skin reflectance of a region of the skin of a subject by supplying a defined amount of energy and irradiating the region of the skin of the subject for a defined period of time and measuring the change of temperature of the skin of the subject (ΔT) during the defined period of time. The present invention further relates to a system for irradiating the skin of a subject comprising: a radiation emitting unit, a radiation energy source connected to the radiation emitting unit, a control unit communicatively connected to the radiation energy source, and a measuring unit for measuring one or more physiological parameters of the subject, further comprising: an user interface configured to store safety and irradiation parameters, wherein the system is configured 1) to interrupt or adjust the energy supply to the radiation emitting unit in case the temperature of the skin of the subject exceeds a maximum temperature, and 2) to execute a radiation regime and wherein the radiation regime is defined such that one or more of the irradiation parameters are taken into account during irradiation.

### DESCRIPTION

The present invention relates to a method of determining the skin reflectance of a region of the skin of a subject and a system for irradiating a region of the skin of a subject with radiation having a wavelength in the range of 300 nm to 1000 nm and use of the system of the present invention in the treatment of a systemic disorder. The present invention further relates to a radiation-emitting device for use in the system according to the present invention, a computer-implemented method for providing an radiation regime for use in the system according to the present invention and correlating computer program and computer-readable medium. The present invention also relates to a method of irradiating a region of the skin of a subject for treating a disease.

Radiation-emitting devices for irradiating a region of the skin of a subject, such as devices used for (medical) phototherapy and devices used in cosmetics, e.g. hair removal devices, tanning devices, tattoo removal devices, are well-known in the art. Typically, devices for irradiating the skin of a subject are provided as radiation-emitting devices applied directly onto the skin of a subject, such as hand-held devices, for irradiating local areas of the skin or radiation-emitting devices located remote from the skin of a subject configured to irradiate a larger area of the skin of a subject, or even configured to perform a full-body irradiation of the skin of a subject. Irrespective the particular use of the device, i.e. either to apply a phototherapy or irradiating the skin of a subject for wellness, cosmetic and/or aesthetic purposes, delivering of the irradiation dose to the subject in an effective, healthy and safe manner is a challenging process dependent on many factors.

For radiation-emitting devices applied directly onto the skin of a subject, providing the correct settings of radiation to irradiate the local area of the skin of the subject is less complex. Examples of such radiation-emitting devices are for example described in United States patent application US 2007/0208395 A1, International patent application WO 2012/011013 A2, and United States patent application US 2017/0165498 A1. In such devices, due to the direct application of the device onto the skin, the complete delivery of the irradiation dose to the subject is safeguarded.

However, for radiation-emitting devices located remote from the skin of a subject for irradiating a larger area of the skin of the subject or even irradiating the full-body of the subject, providing the correct settings of radiation to irradiate the skin of the subject is much more complex and is not only dependent on the specifications of the device used, but also of the subject to be irradiated.

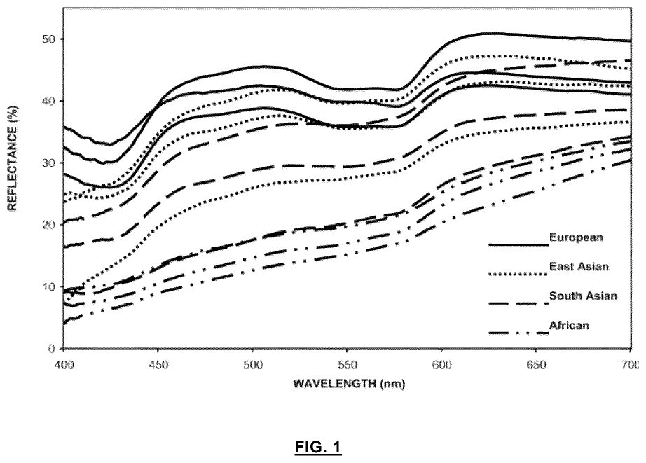

For example, in the field of phototherapy there are currently many phototherapy devices on the market emitting radiation at a defined wavelength that ranges from the ultraviolet to the Infra-Red, therefore including (visible) light (blue, green, yellow and red). An irradiation dose is delivered to the patient that is depending on the intensity of the radiance of the light source (in Joule) and the duration of the radiance. When the surface area of the skin is known, the power density of the irradiation of the skin can be calculated (W/cm²). The energy absorbed by the skin of the subject depends on the radiation dose emitted from the light device, the distance between the irradiated area and the radiation-emitting source, and the skin reflectance. For example for a dark skin type radiation absorption is higher compared to lighter skin type as can be seen in FIG. 1 showing the variation of reflectance by human pigment variation. In some cases, a light skin type can have a reflectance of 40%, meaning only 60% of the total energy radiated from the radiation-emitting source is actually absorbed by the skin of the subject. A recognized tool to define the skin type of a subject is to make use of the Fitzpatrick scale (also referred to as the Fitzpatrick skin typing test or Fitzpatrick phototyping scale) which is a numerical classification schema for human skin colour. Despite the limitations of the applicability of the Fitzpatrick scale, the reflection or absorption of the radiation by the skin not only depends on skin-type, but also on the wavelength of the radiation as well. It is further noted that in most of the cases a treatment time is defined based on the physician experience or clinical guidelines and is fixed independently on the patient skin type.

In addition, the current irradiation approach used in phototherapy (but also used in cosmetics) does not take into account many other relevant variables, such as the variation of the radiation output during the run-up phase and aging-effect (lifetime) of the radiation source applied in the device. Most radiation sources show a run-up behaviour, where the radiation output is either increasing (e.g. CFL/TL/HID light sources) or decreasing (e.g. LED sources) during run-up. Consequently, the time needed to reach a stable radiation output differs per radiation source type.

Another relevant variable to take into account is the aging behaviour causing the radiation output to vary over time. The aging behaviour could result in a decrease over time (e.g. CFL/TL light sources) or result in an initial increase followed by a decrease over time (e.g. LED sources).

For UV therapy, or use of UV for cosmetic/aesthetic purposes, because the risk of an overdose may be harmful, a Minimum Erythema Dose (MED) is established before starting the treatment to avoid skin burn (that is more likely to occur on light skin types). However, by establishing such MED in order to avoid skin burn, the efficacy of the UV therapy is significantly reduced.

Further, even during the process of performing irradiation of the skin of a subject, the skin of the subject may respond differently. For example, (temporary) skin discolorations may occur requiring a different irradiation approach than pre-defined before starting the irradiation method.

Given the complexity of irradiating the skin of a subject in a safe, healthy and effective manner, the present invention now provides for a method of determining the skin type of a subject to be irradiated taken into account the skin reflectance of the subject to be irradiated. In particular, the present invention provides for a method of the continuous determination of the absorbed energy by the skin during irradiation of the skin of the subject. As such, the present invention provides for an automated skin type detection method. The method of the present invention is particularly suitable in use of irradiating a larger area of the skin of the subject or even in use of irradiating the full-body of the subject using a radiation-emitting device located remote from the skin of a subject. In particular, the method of the present invention is particularly suitable in use of a method of irradiating the skin of a subject by using a radiation-emitting device, wherein the radiation-emitting device is positioned such that the radiation-emitting device causes skin reflectance of the radiation dose provided by the radiation-emitting device.

In addition, the present invention also provides for a system wherein the skin of a subject is irradiated taken into account the different relevant parameters of the device itself, the characteristics of the skin of the subject to be irradiated and physiological limits of the subject to be irradiated (as well as regulatory constraints like maximum skin temperatures).

In a first aspect of the invention, the invention relates to a method of determining the skin reflectance of a region of the skin of a subject, wherein the method comprises the steps of:
providing a radiation emitting unit configured to emit radiation having a wavelength in the range of 300 nm to 1000 nm, wherein the radiation emitting unit is further configured to direct the radiation emitted to the region of the skin of the subject;
supplying a defined amount of energy to the radiation emitting unit and irradiating the region of the skin of the subject for a defined period of time (Δt);
measuring the change of temperature of the skin of the subject (ΔT) during the defined period of time (Δt);
calculating the average skin temperature increase (ΔT/Δt); and
determining, based on the average skin temperature increase, the skin reflectance as a function of the amount of absorbed energy by the region of the skin of the subject.

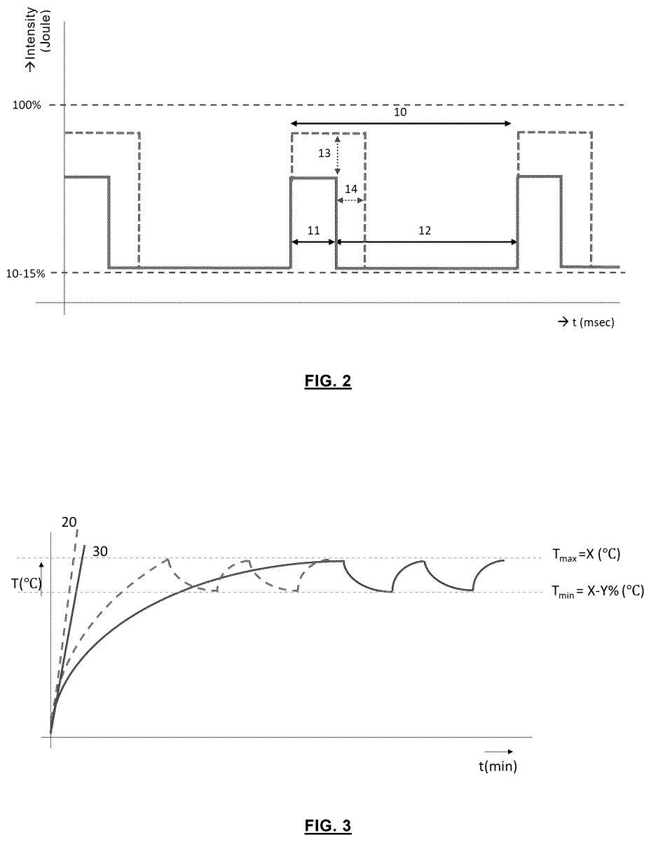

By providing the skin type defining method as provided above, the present invention now provides for a method for irradiating the skin of a subject wherein a highly subject-specific radiation regime can be applied to the subject. An example of such a radiation regime is further exemplified in FIG. 2. By adjusting the radiation regime based on the subject's skin type, i.e. defined by the amount of absorbed energy by the skin of the subject, the method of irradiating the skin of the subject can be further optimised taken into account safety and health aspects in relation to the subject irradiated.

The above method is further exemplified in FIG. 3, wherein the difference between a first skin type 20 (skin type 1) and a second skin type 30 (skin type 2) is determined by measuring and calculating the average skin temperature increase (ΔT/Δt), i.e. the directional coefficient of each of the skin types 20, 30. Subsequently, the radiation regime is adjusted such that the respective skin type 20, 30 is irradiated in such way that, in this particular example, the skin temperature of the subject is kept within a minimum skin temperature (Tₘᵢₙ in FIG. 3) and a maximum skin temperature (Tₘₐₓ in FIG. 3), wherein the minimum skin temperature can be a pre-defined percentage of the maximum skin temperature.

Analogously, by determining the skin type of the subject, the irradiation dose to be delivered to the subject can be determined (and monitored) in a more reliable manner. By defining the skin type of the subject using the skin type determining method of the present invention, the amount of radiation dose reflected by the skin can be calculated based on which the radiation regime, including parameters such as duration of irradiation, irradiation intensity, duty cycle and the like, can be designed based on a personal skin profile.

In addition to providing a personalised, subject-specific radiation regime, the method of the first aspect of the present invention not only provides for an automated skin type detection method, the method of the present invention is also designed such that the method automatically corrects for the radiation output of the radiation emitting unit during the run-up phase and aging-effect (lifetime) of the radiation source applied in the device. Thus, by using the same radiation source for determining the skin type of the subject and for performing the method of irradiating the subject, the method of the present invention provides for a further improved method for delivering an irradiation dose to the subject.

Preferably, the method of the first aspect of the present invention is performed before subjecting the subject to a radiation regime. Additionally or alternatively, the method of the first aspect of the present invention may be performed during the method of irradiating the region of the skin of the subject, i.e. during application of the irradiation dose to the subject. By performing the method of determining the skin type of the subject during irradiation, any changes and variations of the skin type of the subject during such irradiation can be taken into account in further adjusting the radiation regime. Often, during irradiation of a region of the skin of a subject, the skin reflectance of the same region may vary over time during irradiation. By correcting the delivery of irradiation dose in case of variation of skin reflectance, an even more reliable and robust irradiation dose delivery method can be provided using the skin determination method of the present invention.

It is further noted that the skin type determination method of the present invention may be applied to multiple regions of the skin of a subject. By measuring the skin temperature change on different positions on the body of the subject, the method of the present invention may provide for the application of an adjusted radiation regime varying for specific body parts. As such, the radiation regime applied to the subject may be of a dynamic, flexible and controllable radiation type wherein the actual irradiation dose delivered to the subject is monitored over time.

In a second aspect of the invention, the invention relates to a method for irradiating a region of the skin of a subject, wherein the method comprises the steps of:
providing a radiation emitting unit configured to emit radiation having a wavelength in the range of 300 nm to 1000 nm, wherein the radiation emitting unit is further configured to direct the radiation emitted to the region of the skin of the subject;
performing the method of determining the skin reflectance of the region of the skin of the subject according to the first aspect of the present invention; and
irradiating the region of the skin of the subject, by supplying energy to the radiation emitting unit, wherein the supply of energy is adjusted based on the skin reflectance determined.

As explained above, by determining the skin reflectance of the region of the skin of the subject, the present invention provides for an irradiation method wherein the radiation regime, and thus the irradiation dose delivered to the subject, is adjusted based on subject-specific characteristics. By providing such method, changing the radiation settings of the radiation emitting unit is now possible taken into account the specific skin characteristics of the subject and the radiation output of the radiation emitting unit during the run-up phase and aging-effect (lifetime) of the radiation source applied in the device.

In a third aspect of the invention, the invention relates to a system for irradiating a region of the skin of a subject with radiation having a wavelength in the range of 300 nm to 1000 nm, the system comprising:
a radiation emitting unit configured to emit radiation having a wavelength in the range of 300 nm to 1000 nm, wherein the radiation emitting unit is further configured to direct the radiation emitted to the region of the skin of the subject;
a radiation energy source connected to the radiation emitting unit, wherein the radiation energy source is configured to supply an amount of energy to the radiation emitting unit;
a control unit communicatively connected to the radiation energy source and configured to control the supply of energy to the radiation emitting unit by controlling the radiation energy source; and
a measuring unit for measuring one or more physiological parameters of the subject, wherein the physiological parameters measured comprise at least the temperature of the skin of the subject.

The system of the present invention further comprises:
an user interface unit communicatively connectable to the control unit, wherein the user interface unit is configured to receive and/or store:
safety parameters, wherein the safety parameters are correlated to the physiological parameters measured and wherein the physiologically correlated safety parameters comprise at least a pre-defined maximum allowable skin temperature (Tₘₐₓ); and
irradiation parameters, wherein the irradiation parameters are selected from the group consisting of minimum irradiation dose (Dₘᵢₙ), maximum irradiation dose (Dₘₐₓ), irradiation time (tᵢᵣᵣ), preferred minimum skin irradiation temperature (T_{irr,min}) and preferred maximum skin irradiation temperature (T_{irr,max}), said preferred maximum skin irradiation temperature (T_{irr,max}) is equal to or lower than said pre-defined maximum allowable skin temperature (Tₘₐₓ).

In order to provide a system for irradiating a region of the skin of a subject with radiation in a more safe, healthy and effective manner, the system of the present invention is configured to interrupt or adjust the energy supply to the radiation emitting unit in case the temperature of the skin of the subject exceeds said pre-defined maximum skin temperature (Tₘₐₓ), and wherein the system is further configured to execute a radiation regime, wherein the radiation regime comprises a power setting based on which the radiation energy source supplies energy to the radiation emitting unit and wherein the power setting is defined such that one or more of the irradiation parameters are taken into account during irradiation of the region of the skin of a subject.

According to the present invention a system is provided wherein the radiation regime is controlled by the power setting such that the irradiation of the skin of the subject is within one or more (preferably all) irradiation parameters defined in relation to the specific user requirements and/or pre-defined irradiation characteristics. The concept of the radiation regime, i.e. the radiation dose provided by the radiation emitting unit, is depicted in FIG. 2. The power setting of the system of the present invention is defined such that it is able to control the Pulse-With Modulation (PWM) and/or Amplitude Modulation (AM) to control the radiation output. Also the power setting of the system of the present invention is able to control the treatment time in order to control the irradiation dose delivered to the skin of the subject. In other words, the power setting is able to provide a duty cycle which provides the most optimal irradiation of the skin of a subject for a certain purpose, e.g. treating the skin of the subject or the like, taken into account the subject-specific and/or irradiation-specific requirements set. In FIG. 2, the adjustable power settings include the duty cycle 10 defined by the tₒₙ 11 and t_{off} 12. The power setting can be adjusted by changing the AM 13 and PWM 14, thereby defining respectively height and width of the pulse, in order to take into account one or more irradiation parameters for irradiating an area of the skin of a subject.

By providing the system of the present invention the skin of a subject can be irradiated with radiation having a wavelength in the range of 300 nm to 1000 nm in a more efficient and in a more subject-specific manner. For example, the power setting of the radiation regime may be defined such that the temperature of the skin of the subject does not exceed the preferred maximum skin irradiation temperature (T_{irr,max}). By providing such a system, the irradiation of the skin of the subject is irradiated in such way that the irradiation procedure as such is not experienced as uncomfortable by the subject.

As used herein, the term “radiation” may refer to electromagnetic radiation including ultraviolet, infrared and visible light. The term radiation in further combination with the defined wavelength range of 300 nm to 1000 nm is intended to encompass such visible light, ultraviolet and infrared light, whereas other electromagnetic radiation, like gamma, microwaves and radio waves are not part of the invention. As such, the system of the present invention is not only applicable for subjects having a skin disease wherein the system is able to perform a phototherapy (visible light and infrared). The system of the present invention is also relevant for subjects having the skin irradiated for cosmetic and/or aesthetic purposes, like tanning of the skin (ultraviolet).

As used herein, the term “irradiation dose” is a broad term and is generally meant to include, without limitation, absorbed energy per unit mass of tissue. For example, as used herein in some embodiments, an irradiation dose may be the amount of irradiation, or absorbed energy per unit mass of tissue, that is received or delivered during a particular period of time.

It is further noted that the “irradiation parameters” as defined above, further define treatment parameters such as an irradiation treatment dose range (Dₘᵢₙ to Dₘₐₓ), an irradiation treatment time (tₜᵣₑₐₜ) and an irradiation treatment skin temperature range (T_{irr,min} to T_{irr,max}).

It is further noted that the system of the present invention is “configured to execute a radiation regime”. The actual execution of the radiation regime by the system of the present invention may be provided by the control unit of the system (that might be an integral part of the radiation device) and/or by the user interface unit (that might be an integral part of the radiation device, or even a remote device such as a remote computer or smart device).

As used herein, the term “radiation emitting unit” refers to the radiation emitting part of a radiation emitting device, which device may be in the form of a hand-held device (to irradiate specific parts of the skin of a subject) or in the form of a radiation bed-like device, similar to an indoor tanning device, for providing a full body irradiation of the skin of a subject. The radiation emitting unit may be configured to comprise radiation emitting parts, like LEDs, OLED, fluorescent lamps or high-intensity discharge lamps. The radiation emitting parts may be configured to provide and select one or more wavelengths from a broad spectrum of wavelengths. Alternatively, the radiation emitting unit may be configured to provide various wavelengths using different radiation emitting parts, wherein each of the radiation emitting parts is able to provide a specific (narrow) range of wavelengths. The radiation emitting unit may also comprise one or more filters and/or an adjustable filter in order to select the required wavelength for irradiation of the skin of the subject.

As used herein, the term “radiation energy source” refers to an energy source for providing energy to the radiation emitting unit. By adjusting the energy provided to the radiation emitting unit, the radiation intensity can be adjusted accordingly. Therefore, by controlling the radiation energy source of the system of the present invention, the radiation intensity of the radiation emitted from the radiation emitting unit can be controlled in a reliable and reproducible manner.

As used herein, the term “control unit” refers to a unit able to provide instructions to the radiation energy source. Optionally, the control unit of the system may be communicatively connected to the radiation emitting unit directly in order to select of define a relevant wavelength range for the irradiation of the skin of a subject. However, such direct communication is not necessary in case instructions are received from the user interface unit (e.g. a remote computer or smart device) communicatively connectable with the radiation emitting unit.

As used herein, the term “measuring unit” refers to a sensor unit able to measure one or more physiological parameters of the subject. The measuring unit may include many different sensors, wherein each sensor is able to monitor a specific physiological parameter. The measuring unit of the system of the present invention may also comprise a smart device, such as a smart watch worn by the subject to be irradiated, communicatively connectable with the system of the present invention, e.g. the control unit of the system of the present invention.

As used herein, the term “user interface unit” refers to an interface wherein the user or physician/medical practitioner is able to provide subject specific or radiation regime specific input. The user interface unit of the system may be a display communicatively connected or connectable to the control unit of the system. Alternatively, the user interface unit may also be a remote computer or smart device, such as a smart phone or tablet, communicatively connectable to the control unit of the system. Also, in an embodiment of the system of the present invention, the user interface unit may be communicatively connected to the radiation emitting unit and/or measuring unit directly.

As used herein, the term “communicatively connected” or “communicatively connectable” may include the wired coupling of one unit to another unit of the system of the present invention. However, the term may also encompass other types of couplings, such as non-wired couplings, like Bluetooth coupling. The term “communicatively” refers to a one-way or two-way communication of data and/or instructions between different units of the system of the present invention.

In an example of the system of the present invention, in order to increase the efficiency of the irradiation procedure and to minimize the time needed to irradiate the skin of the subject, the power setting of the radiation regime may be defined such that the temperature of the skin of the subject does not fall below the preferred minimum skin irradiation temperature (T_{irr,min}). By monitoring the subject's skin temperature and by providing a radiation regime which complies with the pre-defined skin temperatures set by the subject, the physician, medical practitioner or radiation algorithm, a time-efficient irradiation scheme can be provided which complies with the requirements set by either the subject or other party involved (e.g. radiation algorithm or physician, medical practitioner) in using the radiation system of the present invention.

Given the above, skin temperature is one of the most important physiological parameters as a radiation regime decisive factor in irradiating the skin of a subject. However, other physiological parameters may also play an important role during the irradiation of a subject. As in most cases the subject is lying down, in order to undergo irradiation of the skin in the most feasible and comfortable way, physiological parameters such as blood pressure may play an important role at the end of the radiation process. In case the blood pressure of the subject is, due to the radiation process, dropped under a certain threshold, it will be difficult for the subject to get up straight from the lying position directly after the radiation process. In order to avoid such risky situations and in order to avoid other health risk issues during or directly after irradiation of the skin of the subject, the physiological parameters measured by the measuring unit of the system may be further selected from the group consisting of blood pressure, heart rate, blood oxygen saturation, respiratory rate and skin conductance. In addition to measuring additional physiological parameters by the measuring unit of the system, the user interface is preferably configured to receive and/or store physiologically correlated safety parameters such as a pre-defined allowable blood pressure range, a pre-defined allowable heart rate range, a pre-defined allowable minimum blood oxygen saturation, a pre-defined allowable respiratory rate range and a pre-defined skin conductance range. By providing the physiologically correlated safety parameters the system is able to interrupt or adjust the energy supply to the radiation emitting unit in case at least one physiological parameter measured by the measuring unit deviates from the at least one physiologically correlated safety parameter.

Not only the physiological parameters and correlated safety parameters may be decisive for interrupting or adjusting the energy supply to the radiation unit or to interrupt or adjust the power setting of the radiation regime, also the irradiation parameters may be monitored by the system. Obviously, the radiation regime is finished by the time the irradiation parameters are met, e.g. in case the irradiation dose (D) is delivered to the subject. However, in case, during execution of the radiation regime, at least one of the irradiation parameters is not met, the power setting of the radiation regime is interrupted or adjusted in order to avoid irradiation of the skin of the subject in an ineffective or unhealthy, risky manner.

The power setting of the radiation regime may be defined and adjusted using different radiation parameters known to the person skilled in the art. Preferably, the power setting of the radiation regime is defined based on radiation parameters selected from the group consisting of power setting time (t_{D}), pulse width (P_{width}), maximum radiation intensity (Eₘₐₓ), minimum radiation intensity (Eₘᵢₙ), pulse frequency (P_{freq}), radiation wavelength and radiation waveform, such as a sine wave, a square wave, a triangular wave, a sawtooth wave, a ramp wave or the like. The power setting is preferably defined based on the radiation parameters provided above. However, other radiation parameters (including type of radiation source used, aging effect of the radiation emitting unit, and the like) may also be taken into account in defining a subject specific compliant radiation regime for irradiating the skin of the subject. Even further, the power setting of the radiation regime may be a pulsed radiation energy control loop wherein the intensity and duration of the pulses of radiation energy are balanced based on the physiologically correlated safety parameters and irradiation parameters.

Also, in line with the above, during execution of the radiation regime, the radiation regime or power setting of the radiation regime may be adjusted in case the temperature of the skin of the subject is outside a preferred skin temperature range defined by the preferred minimum skin irradiation temperature (T_{irr,min}) and preferred maximum skin irradiation temperature (T_{irr,max}). Even further, it is noted that the term “defined”, as used herein in relation to defining the power setting or defining a radiation regime, may be used as a synonym for calculated or based on the experience of a physician/medical practitioner. Although the calculation of a power setting balancing the different parameters is preferred, the input provided by a physician/medical practitioner might also result in a highly valuable radiation regime.

In case the system of the present invention comprises radiation-emitting devices located remote from the skin of a subject, the high variability of skin types of different subjects, but also skin type differences for an individual subject, have to be taken into account in order to provide the most optimal irradiating setting for the subject to be treated. In order to correct for any skin type differences, the system of the present invention may be further configured to, before and/or during executing the radiation regime:
supply a defined amount of energy to the radiation emitting unit and irradiating the region of the skin of the subject for a defined period of time (Δt);
measure the change of temperature of the skin of the subject (ΔT) during the defined period of time (Δt);
calculate the average skin temperature increase (ΔT/Δt);
determine, based on the average skin temperature increase, the amount of absorbed energy by the region of the skin of the subject; and
execute the radiation regime wherein the power setting of the radiation regime is adjusted based on the amount of absorbed energy by the region of the skin of the subject determined.

By providing the skin type defining method as provided above, the system now provides for a system for irradiating the skin of a subject with a highly subject-specific radiation regime applied to the subject. By adjusting the power setting of the radiation regime based on the subject's skin type, i.e. defined by the amount of absorbed energy by the skin of the subject, the system of the present invention now provides for a method of irradiating the skin of a subject, and even different regions of the skin of the same subject, in such a way that the most optimal radiation regime can be applied taken into account all safety and health aspects in relation to the specific subject irradiated with the system of the present invention.

The above method is further exemplified in FIG. 3, wherein the difference between a first skin type 20 (skin type 1) and a second skin type 30 (skin type 2) is determined by measuring and calculating the average skin temperature increase (ΔT/Δt), i.e. the directional coefficient of each of the skin types 20, 30. Subsequently, the power settings are adjusted such that the respective skin type 20, 30 is irradiated in such way that the skin temperature of the subject is kept within a minimum skin temperature (Tₘᵢₙ in FIG. 3) and a maximum skin temperature (Tₘₐₓ in FIG. 3), wherein the minimum skin temperature can be a pre-defined percentage of the maximum skin temperature.

In an embodiment of the present invention the system is further configured to select the wavelength of the radiation emitted by the radiation emitting unit. Such selection of the wavelength may be provided by emitting the selected wavelength (or range of wavelengths) using the same source of radiation (e.g. same light source) or may, alternatively be provided by selecting a suitable source of radiation (or combination of sources of radiation) providing the wavelength to be selected by the system.

As the radiation emitting unit is located at some distance from the skin of the subject to be irradiated the distance of the radiation emitting unit to the surface of the skin of the subject may play a further role in the efficiency and efficacy of the irradiation of the skin of the subject. In order to provide a system wherein the distance of the radiation emitting unit to the surface of the skin of the subject is taken into account, the system of the present invention may further comprise:
a distance measuring unit configured to measure the distance of the subject's skin to the radiation emitting unit,

wherein the system is configured to determine whether or not the radiation emitting unit is positioned within a pre-defined preferred distance range.

In case the distance measuring unit determines that the radiation emitting unit is outside the pre-defined preferred distance range, the system may be configured to interrupt or adjust the energy supply to the radiation emitting unit. It is noted that the supply of energy to the radiation emitting unit may be adjusted or interrupted locally, i.e. having an effect on only a part of the radiation emitting unit. In this respect, it is noted that the distance between the radiation emitting unit (normally a flat surface located above the subject lying down) and the surface of the skin of the subject may vary locally, due to the variations and irregularities of the skin of the subject. As a consequence, the radiation emitted by the radiation emitting unit and received by the skin of the subject may have a different intensity at one region of the skin compared to another region of the skin irradiated by the same radiation emitting unit. In order to balance the intensity and to compensate any differences in irradiation intensity, the system may be configured to provide partially adjusted energy supplies to the radiation emitting unit in order to provide a method wherein the irradiation intensity throughout the surface of the skin of the subject irradiated by the system of the present invention is within an accepted (narrow) intensity range.

As already stated above, other relevant variables, such as the variation of the radiation output during the run-up phase and aging-effect (lifetime) of radiation source applied in the system may have an important effect on the radiation emitted by the radiation emitting unit and the subsequent irradiation of the skin of the subject. In order to compensate for any variations in radiation output, the system of the present invention may be configured to determine the maximum stable amount of energy emitted by the radiation emitting unit based on which the radiation regime is adjusted.

Regarding the user interface unit of the system of the present invention, it is noted that the user interface unit may comprise a plurality of pre-defined power settings, e.g. a library of power settings, such as a combination of PWM and AM settings, corresponding to different radiation regimes. The user interface unit may further configured to select a pre-defined power setting closely resembling the power setting defined based on the physiological parameters, physiologically correlated safety parameters and/or irradiation parameters provided before or during execution of the radiation regime.

In a fourth aspect of the present invention, the invention relates to a system for use in the treatment of a skin condition or unpleasant sensory experience present on a region of the skin of a subject, wherein the system is a system according to the present invention. The skin condition may be selected from the group consisting of inflammatory skin conditions, such as eczema, atopic dermatitis, vitiligo, acne, rosacea, pruritus and psoriasis. The unpleasant sensory experience may be selected from the group consisting of pruritus and pain.

In a fifth aspect of the present invention, the invention relates to a system for use in the treatment of a systemic disorder, wherein the system is a system according to the present invention and wherein the systemic disorder is selected from the group consisting of hypertension, hyperbilirubinemia, Raynaud syndrome, Crigler-Najjar syndrome, pain, depression, Chronic Fatigue Syndrome and Long Covid Rehabilitation.

In a sixth aspect of the present invention, the invention relates the non-therapeutic use of the system according to the present invention for irradiating a region of the skin of a subject. Such use may encompass the use of the system of the present invention for the cosmetic treatment of the skin of a subject, including tanning, hair removal, tattoo removal and the like. Another use of the system of the present invention may encompass the use of the system of the present invention for wellness therapy, e.g. relaxation of the muscles of the subject irradiated by the system of the present invention.

In a seventh aspect of the present invention, the invention relates to a radiation-emitting device for use in the system according to the present invention, wherein the radiation-emitting device comprises the radiation emitting unit, the radiation energy source connected to the radiation emitting unit and the control unit connected to the radiation energy source, wherein the measuring unit is an integral unit of the radiation-emitting device. Even further, the radiation-emitting device may optionally comprise the user interface unit. As the user interface unit may also be a remote computing unit, such as a computer, smartphone or tablet, connectable with the radiation-emitting device, the user interface unit is not necessarily an essential part of the radiation-emitting device of the present invention.

In a eighth aspect of the present invention, the present invention relates to a computer-implemented method for providing an radiation regime for use in the system according to the present invention, wherein the computer-implemented method comprises the steps of:
i) instructing the radiation energy source to supply an initial amount of energy to the radiation emitting unit for a defined period of time (Δt);
ii) measuring the change of temperature of the skin of the subject (ΔT) during the defined period of time (Δt);
iii) calculating the average skin temperature increase (ΔT/Δt);
iv) determining, based on the average skin temperature increase of step iii), the amount of absorbed energy by the region of the skin of the subject;
v) providing a power setting for irradiating the region of the skin of the subject;
vi) adjusting the power setting provided in step v) based on the amount of absorbed energy determined in step iv); and
vii) executing the radiation regime using the adjusted power setting of step vi).

By providing the computer-implemented method of the present invention, the invention provides for an automated irradiation process of the skin of a subject wherein the skin type (and/or skin types) of the subject are automatically taken into account based on which the radiation regime is specified.

The computer-implemented method of the present invention may include step v) of providing a power setting for irradiating the region of the skin of the subject, wherein step v) comprises the steps of:
requesting input from the user regarding physiologically correlated safety parameters and irradiation parameters; and
selecting a power setting based on the input provided.

By providing the computer-implemented method as defined above, the present invention provides for a method wherein the physiological specific properties of the subject are taken into account. Possibly, such physiological specific properties of the subject may also include specific user-preferences in order to facilitate the irradiation of the skin of the subject in a highly efficient, but also in an user-friendly way.

Also, the computer-implemented method according to the present invention may further comprise the steps of:
before performing step i), receiving distance data measured by the distance measuring unit;
determining whether or not the radiation emitting unit is positioned within a pre-defined preferred distance range; and
in case the radiation emitting unit is positioned within the pre-defined preferred distance range perform steps i) to vii) or in case the radiation emitting unit is positioned outside the pre-defined preferred distance range a warning signal is generated.

By including distance data, the computer-implemented method of the present invention provides for a method wherein a warning signal is generated in case the distance from the radiation emitting unit to the surface of the skin of the subject changes during irradiation of the skin. For example, by sudden movement of the subject during irradiation of the skin, a warning signal is highly appreciated to warn the subject that such sudden movement (or any movement at all) has an effect on the subject's safety and/or irradiation efficacy.

Also in order to regulate the skin temperature of the subject irradiated by the system of the present invention, in an embodiment, the present invention provides for a computer-implemented method further comprising the steps of:
during step vii), storing skin temperature data received from the measuring unit; and
instructing the radiation energy source to terminate or adjust the supply of energy to the radiation emitting unit in case the temperature of the skin of the subject exceeds the pre-defined maximum skin temperature (Tₘₐₓ).

In a ninth aspect of the present invention, the present invention relates to a computer program comprising instructions which, when the program is executed by a computer, cause the computer to carry out the computer-implemented method, i.e. the fifth aspect of the present invention.

In an tenth aspect of the present invention, the present invention relates to a computer-readable medium having stored thereon the computer program of the sixth aspect of the present invention.

In a eleventh aspect of the present invention, the invention relates to a method of irradiating a region of the skin of a subject for treating a disease, the method comprising the steps of:
a) providing a subject having a disease, such as a skin condition, unpleasant sensory experience or systemic disorder;
b) requesting input from the user regarding physiologically correlated safety parameters and irradiation parameters;
c) selecting a power setting based on the input provided in step b);
d) executing the radiation regime using the power setting of step c); and
e) terminating the radiation regime once a pre-defined total irradiation dose is delivered to the subject.

The above method may be interrupted or adjusted in case the skin temperature of the subject exceeds the pre-defined maximum skin temperature (Tₘₐₓ).

The above method may further comprise the step of:
during step d), adjusting the power setting or selecting another power setting in case at least one of the irradiation parameters is not met, preferably wherein during step d) the power setting is adjusted or another power setting is selected in case the skin temperature of the subject is outside a pre-defined preferred skin temperature range.

### EXAMPLE

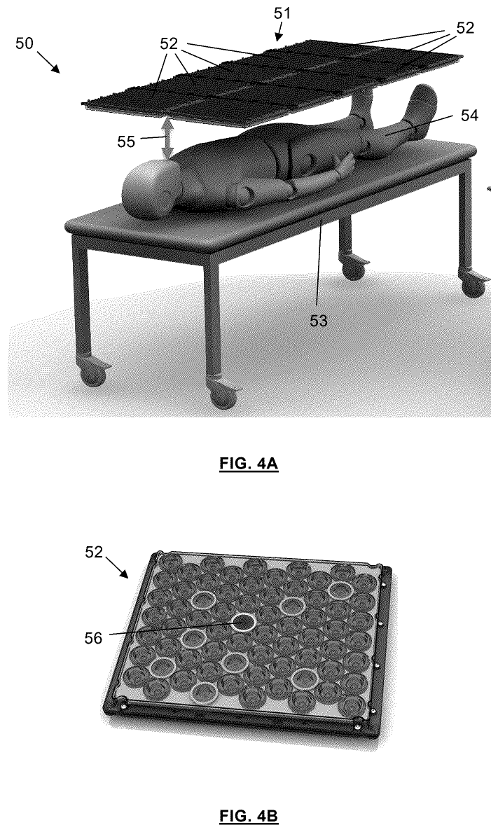

An example of the system according to the present invention is provided in FIG. 4A and FIG. 4B. In FIG. 4A the system 50 of the present invention is shown wherein the system 50 comprises a radiation emitting unit 51, wherein the radiation emitting unit 51 is comprised of multiple subunits 52. The multiple subunits 52 of the radiation emitting unit 51 are in parallel or in series connected to a radiation energy source (not shown) and direct or indirectly to a control unit (not shown). Each of the subunits 52 may be replaceable by another subunit, due to maintenance reasons or even functionality reasons (e.g. replacing one or more subunits 52 having a skin temperature sensor only, with one or more alternative subunits 52 having a skin temperature sensor and, for example, a blood pressure sensor. Also, although multiple subunits 52 are shown in FIG. 4A, the radiation emitting unit 51 may also consist of one large integrated radiation emitting units (not shown) or two large subunits forming a full body radiation emitting unit 51. As shown in FIG. 4A, the system 50 as depicted in FIG. 4A comprises a treatment or therapy table 53 on which the subject 54 is lying. Alternatively, the treatment table 53 can be replaced by a bed or other lying structure suitable for receiving the subject 54. Further indicated in FIG. 4A is the distance 55 between the subject 54 and the radiation emitting unit 51.

In order to measure the skin temperature of the subject 54, the subunits 52 may comprise a skin temperature sensor 56 as depicted in FIG. 4B showing in detail the design of the radiation emitting side of the subunit 52. Other sensors (not shown) may be present as well in order to monitor other physiological parameters of the subject 54.

1. Method of determining the skin reflectance of a region of the skin of a subject, wherein the method comprises the steps of:
providing a radiation emitting unit configured to emit radiation having a wavelength in the range of 300 nm to 1000 nm, wherein the radiation emitting unit is further configured to direct the radiation emitted to the region of the skin of the subject;
supplying a defined amount of energy to the radiation emitting unit and irradiating the region of the skin of the subject for a defined period of time (Δt);
measuring the change of temperature of the skin of the subject (ΔT) during the defined period of time (Δt);
calculating the average skin temperature increase (ΔT/Δt); and
determining, based on the average skin temperature increase, the skin reflectance as a function of the amount of absorbed energy by the region of the skin of the subject.

2. Method according to claim 1, wherein the method is applied to multiple regions of the skin of the subject.

3. Method according to claim 1, wherein the method is performed before irradiating the region of the skin of the subject and/or wherein the method is performed during irradiating the region of the skin of the subject.

4. Method for irradiating a region of the skin of a subject, wherein the method comprises the steps of:
providing a radiation emitting unit configured to emit radiation having a wavelength in the range of 300 nm to 1000 nm, wherein the radiation emitting unit is further configured to direct the radiation emitted to the region of the skin of the subject;
performing the method according to any of the preceding claims; and
irradiating the region of the skin of the subject, by supplying energy to the radiation emitting unit, wherein the supply of energy is adjusted based on the skin reflectance determined.

5. System for irradiating a region of the skin of a subject with radiation having a wavelength in the range of 300 nm to 1000 nm, the system comprising:
a radiation emitting unit configured to emit radiation having a wavelength in the range of 300 nm to 1000 nm, wherein the radiation emitting unit is further configured to direct the radiation emitted to the region of the skin of the subject;
a radiation energy source connected to the radiation emitting unit, wherein the radiation energy source is configured to supply an amount of energy to the radiation emitting unit;
a control unit communicatively connected to the radiation energy source and configured to control the supply of energy to the radiation emitting unit by controlling the radiation energy source; and
a measuring unit for measuring one or more physiological parameters of the subject, wherein the physiological parameters measured comprise at least the temperature of the skin of the subject,
characterised in that the system further comprises:
an user interface unit communicatively connectable to the control unit, wherein the user interface unit is configured to receive and/or store:
safety parameters, wherein the safety parameters are correlated to the physiological parameters measured and wherein the physiologically correlated safety parameters comprise at least a pre-defined maximum allowable skin temperature (Tmax); and
irradiation parameters, wherein the irradiation parameters are selected from the group consisting of minimum irradiation dose (Dmin), maximum irradiation dose (Dmax), irradiation time (tirr), preferred minimum skin irradiation temperature (Tirr,min) and preferred maximum skin irradiation temperature (Tirr,max), said preferred maximum skin irradiation temperature (Tirr,max) is equal to or lower than said pre-defined maximum allowable skin temperature (Tmax),
in that the system is configured to interrupt or adjust the energy supply to the radiation emitting unit in case the temperature of the skin of the subject exceeds said pre-defined maximum skin temperature (Tmax),
and in that the system is configured to execute a radiation regime, wherein the radiation regime comprises a power setting based on which the radiation energy source supplies energy to the radiation emitting unit and wherein the power setting is defined such that one or more of the irradiation parameters are taken into account during irradiation of the region of the skin of a subject.

6. System according to claim 5, wherein the power setting is defined such that the temperature of the skin of the subject does not exceed the preferred maximum skin irradiation temperature (Tirr,max) and, optionally, wherein the temperature of the skin of the subject does not fall below the preferred minimum skin irradiation temperature (Tirr,min).

7. System according to claim 5, wherein the system is further configured to, before and/or during executing the radiation regime:
supply a defined amount of energy to the radiation emitting unit and irradiating the region of the skin of the subject for a defined period of time (Δt);
measure the change of temperature of the skin of the subject (ΔT) during the defined period of time (Δt);
calculate the average skin temperature increase (ΔT/Δt);
determine, based on the average skin temperature increase, the amount of absorbed energy by the region of the skin of the subject; and
execute the radiation regime wherein the power setting of the radiation regime is adjusted based on the amount of absorbed energy by the region of the skin of the subject determined.

8. System according to claim 5, wherein the physiological parameters measured by the measuring unit are further selected from the group consisting of blood pressure, heart rate, blood oxygen saturation, respiratory rate and skin conductance, and wherein the user interface unit is further configured to receive and/or store physiologically correlated safety parameters such as a pre-defined allowable blood pressure range, a pre-defined allowable heart rate range, a pre-defined allowable minimum blood oxygen saturation, a pre-defined allowable respiratory rate range and a pre-defined skin conductance range.

9. System according to claim 5, wherein:
the system is configured to interrupt or adjust the energy supply to the radiation emitting unit in case at least one physiological parameter measured by the measuring unit deviates from the at least one physiologically correlated safety parameter; and/or
during execution of the radiation regime, the power setting of the radiation regime is interrupted or adjusted in case at least one of the irradiation parameters is not met.

10. System according to claim 5, wherein the system further comprises:
a distance measuring unit configured to measure the distance of the subject's skin to the radiation emitting unit,
and wherein the system is configured to determine whether or not the radiation emitting unit is positioned within a pre-defined preferred distance range,
and optionally, wherein the system is configured to interrupt or adjust the energy supply to the radiation emitting unit in case the radiation emitting unit is outside the pre-defined preferred distance range.

11. System according to claim 5, wherein the power setting of the radiation regime is a pulsed radiation energy control loop wherein the intensity and duration of the pulses of radiation energy are balanced based on the physiologically correlated safety parameters and irradiation parameters.

12. System according to any of claim 5, wherein the user interface unit comprises a plurality of pre-defined power settings and wherein a pre-defined power setting is selected closely resembling the power setting defined based on the physiological parameters, physiologically correlated safety parameters and/or irradiation parameters provided before or during execution of the radiation regime.

13. System for use in the treatment of a skin condition or unpleasant sensory experience present on a region of the skin of a subject, wherein the system is a system according to claim 5.

14. System for use according to claim 13, wherein:
the skin condition is selected from the group consisting of inflammatory skin conditions, such as eczema, atopic dermatitis, vitiligo, acne, rosacea, pruritus and psoriasis; and
the unpleasant sensory experience is selected from the group consisting of pruritus and pain.

15. System for use in the treatment of a systemic disorder, wherein the system is a system according to claim 5 and wherein the systemic disorder is selected from the group consisting of hypertension, hyperbilirubinemia, Raynaud syndrome, Crigler-Najjar syndrome, pain, depression, Chronic Fatigue Syndrome and Long Covid Rehabilitation.

16. System of claim 5, wherein the system is used for non-therapeutic irradiating a region of the skin of a subject.

17. System of claim 5, having a radiation-emitting device for use in the system, wherein the radiation-emitting device comprises the radiation emitting unit, the radiation energy source connected to the radiation emitting unit and the control unit connected to the radiation energy source, wherein the measuring unit is an integral unit of the radiation-emitting device, and wherein the radiation-emitting device optionally comprises the user interface unit.

18. Computer-implemented method for providing an radiation regime for use in the system according claim 5, wherein the computer-implemented method comprises the steps of:
i) instructing the radiation energy source to supply an initial amount of energy to the radiation emitting unit for a defined period of time (Δt);
ii) measuring the change of temperature of the skin of the subject (ΔT) during the defined period of time (Δt);
iii) calculating the average skin temperature increase (ΔT/Δt);
iv) determining, based on the average skin temperature increase of step iii), the amount of absorbed energy by the region of the skin of the subject;
v) providing a power setting for irradiating the region of the skin of the subject;
vi) adjusting the power setting provided in step v) based on the amount of absorbed energy determined in step iv); and
vii) executing the radiation regime using the adjusted power setting of step vi).

19. Computer-implemented method according to claim 18, wherein the method further comprises the steps of:
before performing step i), receiving distance data measured by the distance measuring unit;
determining whether or not the radiation emitting unit is positioned within a pre-defined preferred distance range; and
in case the radiation emitting unit is positioned within the pre-defined preferred distance range perform steps i) to vii) or in case the radiation emitting unit is positioned outside the pre-defined preferred distance range a warning signal is generated.

20. Computer-implemented method of claim 18, wherein the method is executed by a computer having a computer program comprising instructions which, when the program is executed by a computer, cause the computer to carry out the method.

21. Computer-implemented method of claim 20, wherein the computer program is stored on a non-transitory computer readable medium.
